(19) United States
(12) Patent Application Publication
(10) Pub. No.: US 2005/0208146 A1
(43) Pub. Date: Sep. 22, 2005

(54) Novel dosage and administration method for oral camptosar

(75) Inventor: Langdon Miller, Lebanon, NJ (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/973,621

(22) Filed: Oct. 25, 2004

Related U.S. Application Data
(60) Provisional application No. 60/515,428, filed on Oct. 30, 2003.

(51) Int. Cl. A61K 31/4745; A61K 9/16

(52) U.S. Cl. 424/490; 514/283

(57) ABSTRACT

The present invention relates to a maximally tolerable dosage of oral irinotecan encapsulated in a semi-solid filling medium which comprises the irinotecan or a derivative thereof; a pharmaceutically acceptable carrier matrix which is a polyglycolized glyceride; and an effective thickening-reducing and stabilizing-promoting amount of one or more pharmaceutically acceptable excipients, where the maximally tolerable oral daily dose is about 60 mg/m² when administered daily for five days every three weeks. The invention also relates to a method of oral administration of irinotecan or a derivative thereof by administering irinotecan, or a derivative thereof, in an encapsulated semi-solid matrix formulation given daily for five days every three weeks. The method is suitable for treatment of cancer in a mammal.

This application claims priority to U.S. Provisional application No. 60/515,428 filed Oct. 30, 2003, the disclosure of which is incorporated into this application in its entirety.

### FIELD OF THE INVENTION

The present invention provides an oral dosage form for camptothecin derivatives, such as, for example, (7-ethyl-10-[4-(1-piperidino)-1-piperidino]-carbonyloxycamptothecin) or its pharmaceutically acceptable salts, especially the hydrochloride (also known as CPT-11; irinotecan; and Camptosar®); or topotecan (9-dimethylaminomethyl-10-hydroxycamptothecin) or pharmaceutically acceptable salts thereof, especially the hydrochloride. The invention also provides a method of use of the camptothecin derivatives for cancer therapy.

### BACKGROUND OF THE INVENTION

Camptothecins are a class of cytotoxic agents which have been undergoing both preclinical and clinical testing against various solid tumors. The nuclear enzyme topoisomerase I (Topo I), along with the other topoisomerases, functions to resolve topological problems during DNA replication. These enzymes are the target for camptothecin and its derivatives. These agents are derivatives of an extract from the Chinese tree Camptotheca acuminata, and were originally shown to be active against L1210 murine leukemia (Wall, M. E., Wani, M. C., Coy, C. E., Palmer, K. H., McPhail, A. T. and Sim, G. A.: Plant antitumor agents. 1. The isolation and structure of camptothecin, a novel alkaloidal leukemia and tumor inhibitor from Camptotheca acuminata, J. Chem. Soc., 88:3888, 1966). Further study confirmed that alkaline labile DNA (single strand) breaks were formed when camptothecin was added to cells in tissue culture and that the breaks rapidly resealed after removal of the drug. These DNA single strand breaks represent the nicks that form when camptothecin stabilizes the covalent adducts between genomic DNA and the reparative nuclear enzyme Topo I (Horwitz, S. B., Chang, C. K. and Grollman, A. P.: Studies on camptothecin. 1. Effects on nucleic acid and protein synthesis. Mol. Pharmacol, 7:632, 1971; Hsiang, Y. H. and Liu, L. F: Identification of mammalian DNA topoisomerase I as an intracellular target of the anticancer drug Camptothecin. Cancer Res., 48: 1722, 1988). Early studies also showed maximal S-phase toxicity, and that the Topo I-associated DNA single strand nicks led to the formation of more persistent double strand breaks which ultimately resulted in cell death. Camptothecins also appear to have other cytotoxic effects which account for their activity in human tumor xenografts that typically have low proportions of cells in S-phase, though these effects are yet to be clearly defined.

A number of more soluble and less toxic analogs of camptothecin have been developed. Among them, Camptosar® (Irinotecan) and Hycamtin® (topotecan hydrochloride) are commercial products.

Topotecan hydrochloride is indicated for the treatment of metastatic carcinoma of the ovary after failure of initial or subsequent chemotherapy and for the treatment of small cell lung cancer sensitive disease after failure of first-line chemotherapy.

Irinotecan was developed in 1983 as a semi-synthetic derivative of camptothecin. The agent has been studied extensively in both preclinical and clinical trials and has shown good anti-tumor activity against a broad spectrum of experimental tumor models (Kunimoto, T., Nitta, K., Tanaka, T. Uchara, N., Baga, H., Takeuchi, M., Yokokura, T., Sawada, S., Miyasaka, T. and Mutai, M.: Antitumor activity of 7-ethyl-10-[4-(1-piperidino)-1-piperidino]carbonyloxycamptothecin against murine tumors. Cancer Res., 47:5944, 1987). The intravenous drug form of irinotecan received FDA approval for the treatment of colon cancer.

Irinotecan is a prodrug which, upon administration to a mammal, is converted to 7-ethyl-10-hydroxycamptothecin (SN-38), by hydrolysis in the liver. SN-38 appears to be the Topo I inhibitor.

It is well known that parenteral administration of antitumor drugs such as, for example, camptothecin derivatives, can be associated with some intrinsic disadvantages and drawbacks, including patient discomfort or the requirement for the patient to travel to the physician's office for drug administration, with obvious results in patient inconvenience. Administration of irinotecan to patients is associated with a variety of side effects that can range from unpleasant to dangerous. Use of irinotecan in excess of the maximum tolerated dose (MTD) can result in unacceptable host toxicity. The toxicity can manifest itself in a number of symptoms, including diarrhea and myelosuppression. Myelosuppression can manifest itself as neutropenic fever or a drop in the absolute neutrophil count to less than 500/mm³. Other measures indicating toxicity include a total white cell count below 2000/mm³, a neutrophil count below 1000/mm³, a hemoglobin level below 8 g/dl, and a platelet count below 100,000/mm³. It is therefore important to maximize the effective dose while minimizing side effects. Irinotecan may have other toxic effects as well. In particular, animal studies suggest that irinotecan is a teratogen.

Thus the need has arisen to develop oral formulations of anti-tumor drugs that would overcome the inconvenience and the discomfort of the patient that are associated with parenteral administration. In addition, there is a need to improve efficacy while ameliorating side effects.

It has long been known in the pharmaceutical industries that capsules are a convenient form for the oral administration of a variety of active agents because of their relative ease of manufacture (compared with other dosage forms such as tablets), flexibility of size and dose. Capsules have traditionally been used for powder or granule formulations but, in recent years, capsules have been adapted to contain the active ingredient in the form of paste, semi-solid or liquid formulations.

This invention addresses the shortcomings of previous methods of dosing and administering irinotecan. It has now been surprisingly found that, by carefully adjusting the dosage of oral irinotecan and administering it orally in a semi-solid matrix the above-mentioned problems can be solved.

### SUMMARY OF THE INVENTION

The invention is directed to the Maximum Tolerated Dose (MTD), dose-limiting toxicities (DLT), pharmacokinetics (PK) and bioavailability of irinotecan and SN-38 from oral administration of semi-solid matrix (SSM) capsules or irinotecan. According to the invention, the MTD and recommended phase 11 starting doses of oral irinotecan or salts or solvates thereof, in an encapsulated semi-solid matrix formulation given daily×5 q 3 weeks are about 50 to 70 mg/m²/day. In one embodiment the dose is 60 mg/m²/day.

In a more preferred embodiment of the invention the oral irinotecan is a pharmaceutically acceptable salt of irinotecan.

In a more preferred embodiment of the invention the oral irinotecan is a hydrochloride salt of irinotecan.

In a most preferred embodiment of the invention the oral irinotecan is irinotecan hydrochloride trihydrate or CPT-11.

In one aspect, the invention comprises an oral dosage form of irinotecan in an encapsulated semi-solid matrix formulation suitable for daily administration at 50 mg/m²/day. One oral dosage form of irinotecan is suitable for administration at an MTD of 60 mg/m²/day. Another oral dosage form is suitable for administration at 70 mg/m²/day. Other, intermediate dosages are also envisioned.

In another aspect, the invention comprises a method of treating a cancer in a mammal. The method comprises oral administration of irinotecan or another salt or solvate or a derivative of irinotecan to the mammal in an amount of about 60 mg/m² daily for five days, preferably consecutive, and repeating the dosing after about three weeks. The preferred form of irinotecan is the hydrochloride salt, as the trihydrate. The irinotecan or derivative of irinotecan is encapsulated in a semi-solid matrix. Pharmaceutically acceptable salts or solvates of irinotecan or a derivative of irinotecan can also be used.

In yet another aspect, the invention comprises a formulation suitable for rapid absorption of irinotecan after oral administration. In one embodiment, the absolute bioavailability of orally administered irinotecan was 25±23% (mean±SD). In another embodiment, the integrated area under the time course curve (AUC) of the active metabolite, SN-38, following oral administration of irinotecan was 50±20% (mean±SD) of the value from an equivalent IV dose, indicating pre-systemic metabolism.

The invention can also comprise a method comprising a 5-day dosing interval of oral irinotecan whereby substantially less systemic exposure to parent irinotecan is produced compared to intravenous (IV) treatment on the weekly×4 q 6-week schedule, while maintaining comparable exposure to SN-38. In one embodiment, the oral route is associated with less irinotecan-related toxicity (e.g., cholinergic syndrome, nausea and vomiting).

### BRIEF DESCRIPTION OF THE FIGURES

FIG. 1 illustrates pharmacokinetic assessment at maximally tolerated dosage.

FIG. 2 illustrates irinotecan concentration (ng/ml) in blood plasma samples as a function of time after administration of intravenous or oral irinotecan.

FIG. 3 illustrates SN-38 concentration (ng/ml) in blood plasma samples as a function of time after intravenous or oral administration.

### DETAILED DESCRIPTION OF THE INVENTION

Terms and acronyms not defined elsewhere include the following. AST is aspartate transaminase, the levels of which in the blood measure liver function. XRT indicates radiation therapy. CNS means central nervous system. GGT is gamma glutamyl transferase, another measure of liver function. The notation “q” as in daily×5 q 3 weeks is read to mean that one full (or several split) dose is administered within a 24 hour period and repeated, preferably on the four consecutive and subsequent days, then after three weeks the 5 days of treatment are repeated. ULN is the upper limit of the normal range. A cycle of administration, in the context of the invention, means administration of about five oral doses of irinotecan over the course of three weeks. Preferably the doses are administered on consecutive days.

Body surface area (BSA) can be measured or can be calculated from any formula standard in the art. One such formula is that of DuBois and DuBois. (A formula to estimate the approximate surface area if height and weight be known, Arch. Int. Med. 17:863 (1916).) A simpler formula is BSA (m²)=0.1173×(body weight (kg))^{0.6466}. (Livingston and Lee, Body surface area prediction in normal-weight and obese patients, Am. J. Physiol. Endocrinol. Metab. 281:E586 (2001).)

Oral administration of irinotecan can improve the safety and convenience of achieving protracted exposure of tumor cells to SN-38. Protracted exposure of the tumor cells to SN-38 as the cells pass through the cell division cycle increases the likelihood that tumor cells in S-phase will be exposed to an effective concentration of active topoisomerase inhibitor.

One advantage of the method of the invention is an improved balance between toxicity to the tumor and toxicity to the host. Indeed, the method of the invention permits administration of irinotecan at or near the MTD for about five days and repetition of the five day treatment every three weeks. Specifically, the invention has identified an oral dose of 60 mg/m²/day as particularly advantageous.

Other doses can also be effective. The oral dose can be less than 52 mg/m². In one embodiment, the oral dose is between about 50 and 52 mg/m², 52 and 54 mg/m², between 54 and 56 mg/m², between 56 and 58 mg/m², between 58 and 60 mg/m², between 60 and 62 mg/m², between 62 and 64 mg/m², between 64 and 66 mg/m², between 66 and 68 mg/m² or between 68 and about 70 mg/m². In another embodiment, the oral dose is at least 68 mg/m². In one preferred embodiment, the oral dose is at least 60 mg/m².

The invention can comprise an oral dosage form of irinotecan comprising irinotecan in an encapsulated semi-solid matrix formulation suitable for daily administration. The semi-solid matrix can comprise lecithin. Alternatively, or in addition, the semi-solid matrix can comprise a polyglycolized glyceride, for example Gelucire 44/14, Gelucire 50/13, Gelucire 35/10, or Gelucire 46/07, or combination thereof. In one preferred embodiment the semi-solid matrix can comprises Gelucire 44/14. Any pharmaceutically acceptable salt form of irinotecan is acceptable.

The invention can also comprise a method of treating a cancer in a mammal comprising oral administration to the mammal, of irinotecan or derivatives, salts or solvates thereof, encapsulated in a semi-solid matrix in an amount of 60 mg/m² daily for five days in a period of about three weeks. In one aspect of the invention, the irinotecan is administered for five consecutive days. Furthermore, the three week cycle of administration can be repeated as needed.

The invention also comprises a method of treating a cancer in a mammal comprising sequential administration of
(i) a therapeutically effective intravenous dose of irinotecan and
(ii) an oral dose of about 60 mg/m² of irinotecan encapsulated in a semi-solid matrix daily for five days. The oral administration can be repeated after about three weeks. The sequential administration can be in any order. Thus, the oral dose can be administered before, or after the intravenous dose.

In one aspect, the mammal can be a human. The human can, moreover, be an adult.

Furthermore, the cancer can be a solid tumor. The invention can also comprise treating a patient having a cancer wherein the cancer is selected from the group consisting of small cell lung cancer, esophageal cancer, kidney cancer, pancreatic cancer, melanoma, bladder cancer, breast cancer, colon cancer, liver cancer, lung cancer, sarcoma, stomach cancer, cholangiocarcinoma, mesothelioma, or prostate cancer.

Additional cancers may also be treated by the methods of the invention, including gastric cancer, metastatic breast cancer, glioblastoma, rectal cancer, multiple myeloma, colorectal, non-small cell lung cancer (NSCLC), bladder cancer, and ovarian cancer.

The method of the invention is suitable for the treatment of several specific types of cancer. In a preferred embodiment the cancer is small cell lung cancer. In another preferred embodiment the cancer is esophageal cancer. In yet another preferred embodiment the cancer is kidney cancer. In still another preferred embodiment the cancer is pancreatic cancer. In yet still another preferred embodiment the cancer is melanoma. Moreover in one preferred embodiment the cancer is bladder cancer. In another preferred embodiment the cancer is breast cancer. In yet another preferred embodiment the cancer is colon cancer. In still another preferred embodiment the cancer is liver cancer. In yet still another preferred embodiment the cancer is lung cancer. The method of the invention is advantageously applied to a cancer wherein the cancer is sarcoma. In another preferred embodiment the cancer is stomach cancer. In yet another preferred embodiment the cancer is cholangiocarcinoma. In still another preferred embodiment the cancer is mesothelioma. In yet still another preferred embodiment the cancer is prostate cancer. The cancer to be treated can also be a gastric cancer. In another preferred embodiment the cancer is metastatic breast cancer. In yet another preferred embodiment the cancer is glioblastoma. In still another preferred embodiment the cancer is rectal cancer. In yet still another preferred embodiment the cancer is multiple myeloma. Furthermore, in one preferred embodiment the cancer is colorectal cancer. In another preferred embodiment the cancer is non-small cell lung cancer. In yet another preferred embodiment the cancer is bladder cancer. In still another preferred embodiment the cancer is ovarian cancer.

In one aspect, the invention comprises a kit comprising at least one effective dose of irinotecan encapsulated in a semi-solid matrix for oral administration and a label specifying that a dose is to be administered orally daily for five consecutive days and then the treatment repeated after three weeks.

In another aspect, the invention comprises a method of effecting protracted SN-38 exposure in a mammal in need thereof comprising oral administration to the mammal, of irinotecan or a derivative, salt, or solvate thereof, encapsulated in a semi-solid matrix in an amount suitable for administration of a dose of about 60 mg/m² daily for five days and repeating the cycle after three weeks, whereby SN-38 is formed metabolically from the irinotecan.

Moreover, the antitumor efficacy of irinotecan may be increased by effecting prolonged exposure to SN-38, the active metabolite. Daily oral dosing can achieve protracted SN-38 exposure without the need for protracted infusions. Oral administration of irinotecan can offer safety and convenience advantages. The oral route of delivery can be preferable in certain treatment settings (e.g., with other oral agents, or with radiotherapy).

### EXAMPLES

### Example 1

### Selection and Treatment of Patients

The following protocol was used to evaluate oral administration of irinotecan. Patient inclusion and exclusion criteria are listed in Tables 1 and 2, respectively. The protocol had two stages, Stage A directed to dose escalation and Stage B directed to pharmacokinetics and bioavailability measurement. Patients were treated as in Table 3. Sequential groups of patients received once daily administration of irinotecan for 5 consecutive days every three weeks. MTD is defined as the dose level below which 2/3 or preferably 2/6 patients experience DLT. To obtain bioavailability data, 24 additional patients were treated at the MTD with alternating IV/oral first-dose administration in Cycles 1 and 2.

TABLE 1
Patient Inclusion Criteria

| Inclusion Factor | Criterion |
|---|---|
| Solid Tumor | Histologically confirmed |
| ECOG PS | 0, 1 or 2 |
| Creatinine | ≦2.0 mg/dl |
| ANC | ≧2,000/μL |
| Hemoglobin | ≧9.0 g/dl |
| Platelet ≧ | 150,000/μL |
| Bilirubin | <ULN |
| AST | ≦3 × ULN |
| | (≦5x if liver metastases present) |
| Age | ≧18 years |
| Informed consent | Able to provide |

TABLE 2
Patient Exclusion Criteria

| Exclusion Factor | Criterion |
|---|---|
| Pregnant or lactating | excluded |
| Prior irinotecan, infusional 5- | excluded |
| FU or capecitabine, | |
| mitomycin or nitrosoureas | |
| 2 prior chemotherapy | excluded |
| regimens | |
| Prior XRT >25% of bone | excluded |
| marrow | |
| Malabsorption, diarrhea, | excluded |
| inflammatory bowel disease, | |
| bowel obstruction, or total | |
| colectomy | |
| Known CNS tumor | excluded |
| involvement | |
| Anticonvulsants | excluded, if within 1 week |
| | prior to starting therapy |

TABLE 3
Methods of Patient Treatment

| Study Element | Criteria |
|---|---|
| Study design: | Stage A: Dose-escalation to MTD |
| | Stage B: PK and bioavailability at MTD |
| Treatment: | Oral capsule once daily × 5 days q 3 weeks |
| Dose levels: | 50, 60, 70 mg/m²/day |
| Supportive care: | Antiemetics, antidiarrheals |
| Dose escalation: | Successive cohorts of 3-6 patients |
| MTD definition: | Highest dose level with < 2/6 DLTs |
| DLT definition: | Hematologic toxicity: Grade 4 neutropenia, |
| | neutropenic fever, |
| | neutropenic infection or Grade 4 |
| | thrombocytopenia |
| | Grade ≧3 diarrhea |
| | Grade ≧2 nausea or vomiting |
| | Other grade ≧3 non-hematologic toxicities |
| | Failure to complete a treatment course |
| | Failure to recover to grade ≦1 toxicity by |
| | day 35 |

One aspect of the invention thus comprises evaluation for therapeutic use of a single-agent oral irinotecan semi-solid matrix formulation given daily for five consecutive days every three weeks. The evaluation includes determination of the MTD and the DLT; the overall safety profile; and the PK and absolute bioavailability of oral irinotecan. In addition, evidence of antitumor activity can be evaluated.

The patients had the characteristics described in Table 4. A group of 43 patients with solid tumors were evaluated. The patients had a median age of 63, with an age range of 29-86. The ratio of men to women (M/F) was 26/17. At the beginning of the trial the patients had Eastern Cooperative Oncology Group Performance Status (ECOG PS) scores of 0/1/2=6/32/5.

TABLE 4
Patient Characteristics

| | Stage A | Stage B |
|---|---|---|
| | (Dose-escalation) | (PK and Bioavailability) |
| Characteristic | N = 19 | N = 24 |
| Age | Median 63 | Median 63 |
| | Range 50-86 | Range 29-84 |
| Sex (Male/Female) | 14/5 | 12/12 |
| ECOG Performance | 4/12/3 | 2/20/2 |
| Status (0/1/2) | | |
| Prior Chemotherapy | Median 1 | Median 1 |
| Regimens | Range 0-2 | Range 0-2 |
| Tumor Types* (n) | Kidney (6), | Lung (6; including 2 |
| | pancreas (2), | SCLC), colon (4), pancreas |
| | melanoma (2), | (3), liver (2), kidney (2), |
| | bladder, abdominal | bladder, |
| | unknown, breast, | cholangiocarcinoma, |
| | colon, esophagus, | melanoma, mesothelioma, |
| | liver, lung, | prostate, sarcoma, |
| | sarcoma, stomach | stomach |

*Listed in descending order of frequency

Oral irinotecan can be formulated as follows. The drug product oral irinotecan was supplied in hard gelatin capsules containing 5, 20, or 50 mg as irinotecan hydrochloride trihydrate in a semi-solid matrix. The composition of the 5, 20, and 50 mg capsules is reported in Table 5.

TABLE 5
Nominal Composition of the Oral Irinotecan Formulation

| | | | | | | | Composition |
|---|---|---|---|---|---|---|---|
| Components | 5 mg | | 20 mg | | 50 mg | | (%) (w/w) |
| Irinotecan | 5.0 | mg | 20.0 | mg | 50.0 | mg | 7.9 |
| hydrochloride | | | | | | | |
| trihydrate (CPT-11) | | | | | | | |
| Lauroyl | 52.4 | mg | 209.6 | mg | 524.0 | mg | 83.2 |
| Macrogolglycerides, | | | | | | | |
| Ph.Eur. (Gelucire) | | | | | | | |
| Lecithin, USP | 5.6 | mg | 22.4 | mg | 56.0 | mg | 8.9 |
| (Epikuron) | | | | | | | |
| Total | 63.0 | mg | 252.0 | mg | 630.0 | mg | 100.0 |
| Capsule size | 2 | | 2 | | 0 | | |

It is important to note that the quantitative compositions are exactly proportional, in other words the percent composition is the same for all capsule strengths.

To differentiate the 5, 20, and 50 mg capsules a colored band was applied to the external surface of the capsule shell (i.e., the colored band will not be in direct contact with the capsule content), namely:
5 mg dosage, Size 2, self-locking hard gelatin capsules Licaps® type, with an opaque white body and cap.
20 mg dosage, Size 2, self-locking hard gelatin capsules, Licaps® type, with an opaque white body and an opaque white-red printed banded cap.
50 mg dosage, Size 0, self-locking hard gelatin capsules, Licaps® type, with an opaque white body and an opaque white-black printed banded cap.
   The drug product was stored at controlled room temperature in opaque white HDPE bottles, closed with child proof, tamper-evident plastic screw cap.

The patients received starting doses of 50 (n=7), 60 (n=32), or 70 (n=4) mg/m²/d. During the dose escalation segment of the trial, DLTs were observed in 1/7 pts at 50 mg/m² as follows: (G3 diarrhea, G3 nausea, G3 dehydration), 0/8 pts at 60 mg/m², and 2/4 pts at 70 mg/m² (G3 diarrhea, G4 ANC [absolute neutrophil counts]; G4 diarrhea, fever, dehydration, fatigue). See also Tables 6 and 7.

TABLE 6
First Cycle DLT's

| | | | | | Total Cycles |
|---|---|---|---|---|---|
| Dose | | Evaluable | Pts with | | Administered |
| (mg/m²/day) | Treated | for DLT | DLT | DLT | (median, range) |
| Stage A | | | | | |
| 50 | 7 | 7 | 1 | Gr. 3 Diarrhea | 18 (2, 2-5) |
| | | | | Gr. 3 Dehydration | |
| | | | | Gr. 3 Nausea | |
| 60 | 8 | 8 | 0 | None | 68 (5, 2-18) |
| 70 | 4 | 4 | 2 | Pt 1: gr. 3 Diarrhea, | 6 (1, 1-2) |
| | | | | gr. 3 Febrile | |
| | | | | Neutropenia | |
| | | | | Pt 2: gr. 4 Diarrhea, | |
| | | | | gr. 4 Dehydration | |
| Stage B | | | | | |
| 60 | 24 | 24 | 7 | Pt 1: Gr. 3 Diarrhea | 79 (2, 1-18) |
| | | | | Pt 2: Gr. 3 Dehydration | |
| | | | | Pt 3: Gr. 3 Diarrhea, Gr. 2 | |
| | | | | Nausea and Vomiting | |
| | | | | Pt 4: Gr. 3 Nausea and | |
| | | | | Vomiting | |
| | | | | Pt 5: Gr. 3 Increase in | |
| | | | | ALT/GGT | |
| | | | | Pt 6: Gr. 3 Diarrhea, Gr. 3 | |
| | | | | Nausea and Vomiting | |
| | | | | Pt 7: Gr. 3 Diarrhea | |

TABLE 7
Grade 3 and 4 Toxicities

| Dose | | | | Neutro- | Neutropenic | Vomit- |
|---|---|---|---|---|---|---|
| (mg/m²/day) | N | Diarrhea | Nausea | penia | Fever | ing |
| Cycle 1 | | | | | | |
| 50 | 7 | 14% | 14% | — | — | |
| 60 | 32 | 13% | 6% | 3% | — | 9% |
| 70 | 4 | 50% | — | 25% | 25% | — |
| All Cycles | | | | | | |
| 50 | 7 | 14% | 14% | — | — | — |
| 60 | 32 | 16% | 9% | 9% | 3% | 12% |
| 70 | 4 | 50% | 3% | 25% | 25% | 3% |

The pharmacokinetic data for irinotecan and SN-38 are presented in Table 8.

TABLE 8
Pharmacokinetics

| | Parameter | | | |
|---|---|---|---|---|
| | Irinotecan | | SN-38 | |
| | mean ± SD | | mean ± SD | |
| | (median, range) | | (median, range) | |
| | IV | PO | IV | PO |
| Dose = 60 mg/m² | N = 14¹ | N = 14¹ | N = 14¹ | N = 14¹ |
| Cₘₐₓ (ng/ml) | 627 ± 142 | 73.1 ± 67.6 | 15.94 ± 7.83 | 7.17 ± 3.90 |
| | (593, 416-897) | (60.1, 9.49-274) | (13.8, 7.26-36.9) | (3.91, 1.31-14.8) |
| tₘₐₓ (h) | End of 1.5-h | 3.15 ± 2.00 | 2.18 ± 1.99² | 2.97 ± 2.21 |
| | infusion | (3.0, 1.0-10.0) | (0.5, 0-2.0) | (2, 1.0-10.0) |
| t_{1/2} (h) | 10.31 ± 2.19 | 9.35 ± 3.24 | 22.95 ± 19.41 | 14.2 ± 5.87 |
| | (10.1, 7.30-17.3) | (9.05, 3.53-17.6) | (19.31, 8.63-94.9) | (13.4, 4.7-25.7) |
| AUC_{0-inf} (ng h/ml) | 3016 ± 830 | 732 ± 665 | 167 ± 77.8 | 84.1 ± 54.7 |
| | (2613, 1722-4788) | (619, 167-2497) | (169, 55.1-358) | (70.7, 22.8-223.6) |

¹Patients treated with both IV and PO irinotecan in a cross-over design.
²Measured from start of 1.5-h infusion.

With oral administration, half-lives were 9.35±3.24 hr (irinotecan) and 14.2±5.87 hr (SN-38). See Table 8. The glucuronidation ratio (SN-38G AUC/SN-38 AUC) was similar for oral (4.2±2.4) and IV dosing (4.7±2.5). See Table 9. The time to the maximum irinotecan concentration was 3.0±2.2 hours. The mean±SD (range) bioavailability for irinotecan was 0.24±0.23 (0.071-0.960) in 17 PK-evaluable patients. The PO/IV ratio of SN-38 AUC was 0.504±0.195 (0.257-0.945), indicating extensive pre-systemic metabolism. See Table 10.

TABLE 9
Metabolic Ratios

| | Parameter | |
|---|---|---|
| | IV | PO |
| dose = 60 mg/m² | mean ± SD | mean ± SD |
| N = 14¹ | (median, range) | (median, range) |
| Metabolic Ratio | 0.056 ± 0.023 | 0.14 ± 0.07 |
| (Ratio of SN-38 AUC/ | (0.051, 0.020-0.101) | (0.13, 0.054-0.319) |
| irinotecan AUC) | | |
| Glucuronidation Ratio | 4.70 ± 2.46 | 4.22 ± 2.40 |
| (Ratio of SN-38G | (5.04, 1.40-9.65) | (3.79, 1.15-9.24) |
| AUC/SN-38 AUC) | | |

¹Patients treated with both IV and PO irinotecan in a cross-over design.

TABLE 10
Bioavailability of Irinotecan

| | Absolute bioavailabiity of | |
|---|---|---|
| | irinotecan | PO/IV SN-38 AUC Ratio |
| Factor | (N = 14) | (N = 14) |
| Mean ± SD: | 0.238 ± 0.231 | 0.504 ± 0.195 |
| Median (range): | 0.154 (0.071-0.960) | 0.447 (0.257-0.945) |

Two confirmed partial responses (PRs) were observed, which were esophageal adenocarcinoma and small cell lung carcinoma (SCLC).

The following conclusions were drawn from the study. The recommended phase 11 dose for semi-solid matrix (SSM) oral irinotecan is 60 mg/m²/daily for 5 days every 3 wks. This dose and schedule maintains SN-38 drug concentrations at a tumor cell cytotoxic level continuously over a 5-day period in each 3-week cycle. The method provides, moreover, acceptable bioavailability of irinotecan and SN-38 and there is evidence of pre-systemic metabolism. See also Table 11.

TABLE 11
Comparison of Exposure over 6-week Period

| | Oral Irinotecan* | IV Irinotecan** |
|---|---|---|
| N | 14 | 99 |
| Irinotecan | | |
| Cₘₐₓ (ng/ml) | 73.1 | 1,500 |
| AUC_{0-inf} (ng h/ml) | 7320 | 42,000 |
| SN-38 | | |
| Cₘₐₓ (ng/ml) | 7.2 | 28 |
| AUC_{0-inf} (ng h/ml) | 841 | 1,068 |

*Daily × 5 q 3 weeks × 2 cycles
**Weekly × 4 q 6 weeks × 1 cycle

The best overall tumor responses were two partial responses: esophageal cancer and small-cell lung cancer cases. Moreover thirteen stable disease cases were found, having a variety of tumor types.

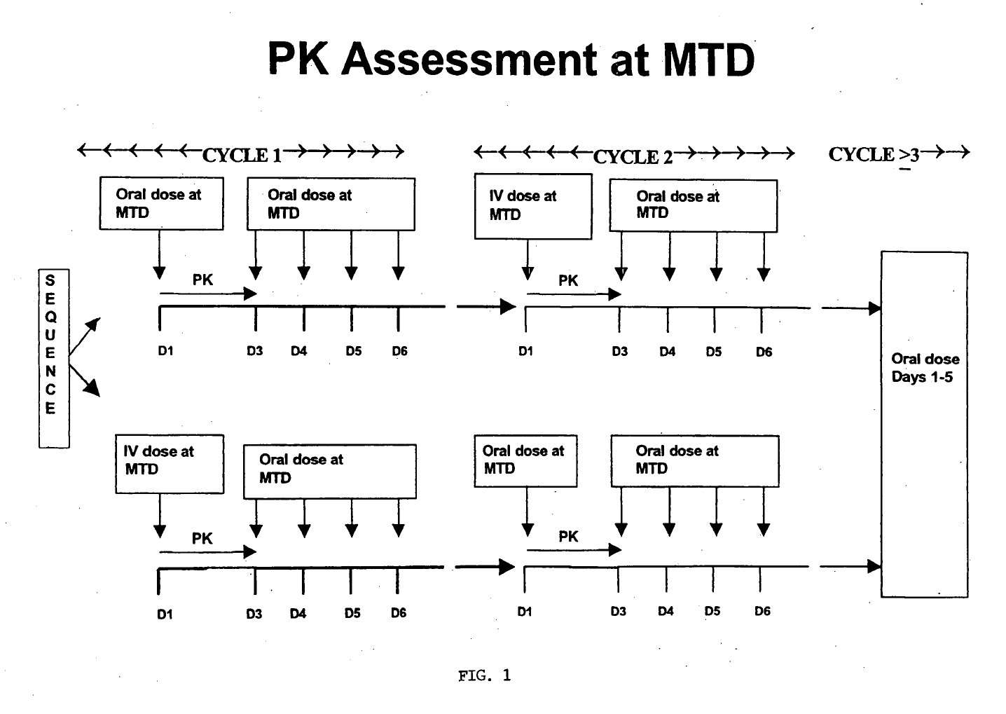

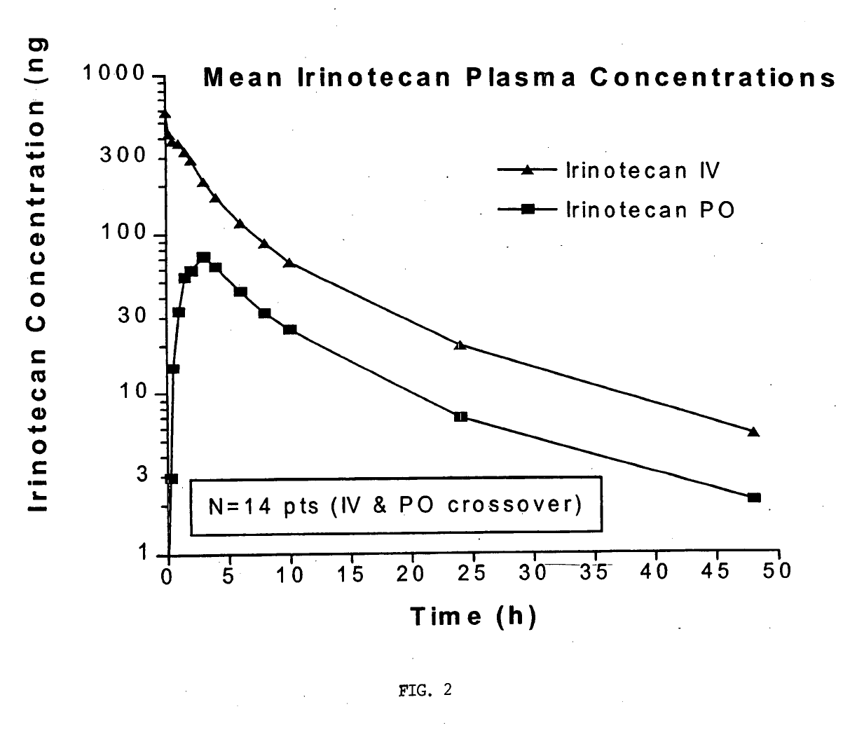

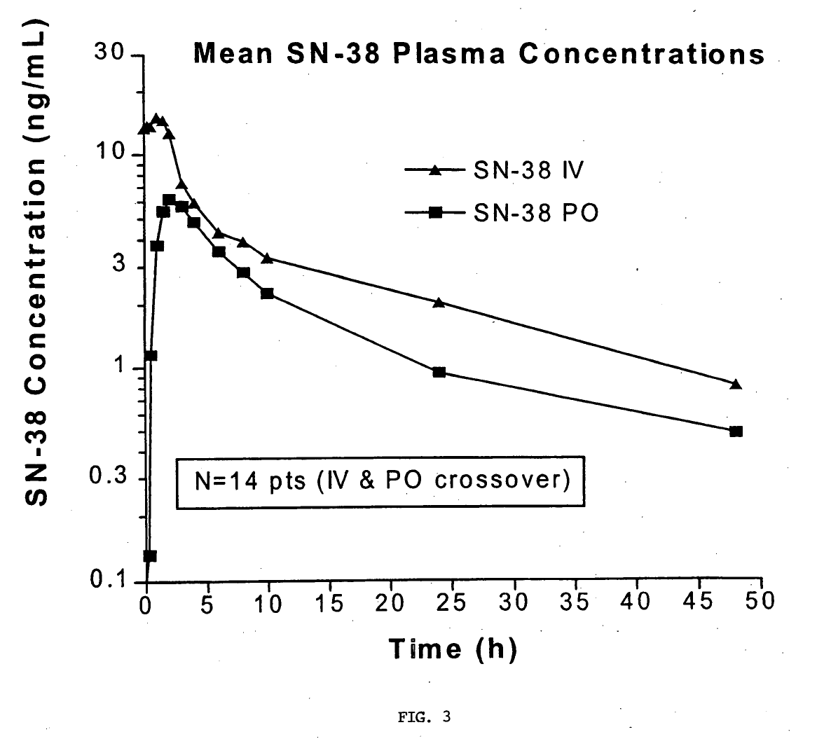

Pharmacokinetics were assessed at MTD as illustrated in FIG. 1. Irinotecan concentration (ng/ml) in blood plasma samples as a function of time after administration of intravenous or oral irinotecan can be ascertained from FIG. 2. SN-38 concentration (ng/ml) in blood plasma samples as a function of time after intravenous or oral administration can be ascertained from FIG. 3.

### Example 2

### Method of Preparation

For each preparation a proper quantity of the selected Gelucire was melted at 60° C. under magnetic stirring. The required amount of melted Gelucire (5 mL) was withdrawn by means of a manual pipette (e.g. Brand-Transferpettor or the like) and added to the required quantity of irinotecan (500 mg). The drug was dispersed in the molten matrix under magnetic stirring at 60° C. for two hours. The dispersion obtained by the above process was then filled into size 0 hard gelatin capsules (0.5 mL/capsule) using a manual pipette.

The capsules manufactured as described above were tested for dissolution rates according to USP Basket method under the conditions of rotating at 100 rpm at 37 C in simulated gastric fluid pH 1.2 without enzymes.

The release profiles of irinotecan from different Gelucire based systems are shown in Table 12. Each Gelucire type is identified by two numbers, termed in the aggregate the Hydrophilic-Lipophilic Balance (HLB) value. The hydrophilicity value is given by the second figure in the HLB. A high hydrophilicity value corresponds to highly hydrophilic excipients.

The results are expressed as percent of the active agent released from the formulation vs. the theoretical as a function of time. The composition of each formulation was basically 50 mg of irinotecan dispersed in 0.5 ml of the appropriate Gelucire per capsule. It is clear from Table 12 that the release of irinotecan is faster from more hydrophilic excipients.

TABLE 12
Rate of Irinotecan Release from Various Formulations

| | % CPT-11 RELEASED | | | |
|---|---|---|---|---|
| | (percent of the theoretical) | | | |
| TIME | Gelucire ® | Gelucire ® | Gelucire ® | Gelucire ® |
| (minutes) | 44/14 | 50/13 | 35/10 | 46/07 |
| 15 | 29.51 | 0.53 | 1.20 | 0.0 |
| 30 | 74.82 | 2.15 | 2.68 | 0.21 |
| 60 | 87.76 | 4.33 | 7.15 | 0.53 |
| 120 | 91.73 | 9.78 | 17.72 | 1.07 |
| 180 | 92.53 | 15.94 | 26.60 | 1.64 |

The disclosure of methods of preparation of the formulation, WO01/30351 is incorporated by reference herein, in its entirety.

An alternative oral form can be found in U.S. Pat. No. 6,569,452, which is incorporated in its entirety by reference herein.

1. An oral dosage form of irinotecan comprising irinotecan in an encapsulated semi-solid matrix formulation suitable for oral administration at 50 to 70 mg/m²/day.

2. The oral dosage form of claim 1, wherein the irinotecan is a hydrochloride salt of irinotecan.

3. The oral dosage form of claim 2, wherein the irinotecan is irinotecan hydrochloride trihydrate or CPT-11.

4. The oral dosage form of claim 1, wherein the semi-solid matrix comprises a lecithin.

5. The oral dosage form of claim 1, wherein the semi-solid matrix comprises a polyglycolized glyceride.

6. The oral dosage form of claim 5, wherein the semi-solid matrix further comprises a lecithin.

7. The oral dosage form of claim 6, wherein the formulation is suitable for oral administration at 50 mg/m²/day.

8. The oral dosage form of claim 6, wherein the formulation is suitable for oral administration at 60 mg/m²/day.

9. The oral dosage form of claim 6, wherein the formulation is suitable for oral administration at 70 mg/m²/day.

10. An oral dosage form of irinotecan comprising about 8% by weight of CPT-11, about 83% by weight lauroyl macrogolglyceride, and about 9% by weight lecithin, suitable for oral administration at about 60 mg/m²/day.

11. A method of treating a cancer in a mammal comprising oral administration to the mammal of CPT-11 encapsulated in a semi-solid matrix formulation in an amount of about 60 mg/m² daily for five days in a period of about three weeks.

12. The method of claim 11 wherein the mammal is a human.

13. The method of claim 12 wherein the human is an adult.

14. The method of claim 11 wherein the cancer is a solid tumor.

15. The method of claim 11 wherein the cancer is selected from the group consisting of small cell lung cancer, esophageal cancer, kidney cancer, pancreatic cancer, melanoma, bladder cancer, breast cancer, colon cancer, liver cancer, lung cancer, sarcoma, stomach cancer, cholangiocarcinoma, mesothelioma, prostate cancer, gastric cancer, metastatic breast cancer, glioblastoma, rectal cancer, multiple myeloma, colorectal, non-small cell lung cancer (NSCLC), bladder cancer, and ovarian cancer.

16. The method of claim 11 wherein the five days are consecutive.

17. The method of claim 11 further comprising repeating the oral administration of irinotecan for another five days.

18. The method of claim 11 further comprising administration of a therapeutically effective intravenous dose of irinotecan.

19. The method of claim 18 wherein the oral dose is administered after the intravenous dose.

20. A method of effecting prolonged SN-38 exposure in a mammal in need thereof comprising oral administration to the mammal, of irinotecan or salt or solvate thereof, encapsulated in a semi-solid matrix in an amount suitable for administration of a dose of about 60 mg/m² daily for five days in a period of about three weeks, whereby SN-38 is formed metabolically from the irinotecan or salt or solvate thereof.

21. The method of claim 20 wherein a metabolic ratio of SN-38 to irinotecan is at least about 0.14.

22. A kit comprising at least one effective dose of irinotecan encapsulated in a semi-solid matrix and a label specifying that one effective dose is to be administered orally daily for five consecutive days and the cycle repeated after three weeks.
